(19) United States
(12) Patent Application Publication
(10) Pub. No.: US 2015/0039241 A1
(43) Pub. Date: Feb. 5, 2015

(54) HYDROCARBON MODELLING

(71) Applicant: INFOCHEM COMPUTER SERVICES LTD., London (GB)

(72) Inventor: Roland A. S. Moorwood, Surrey (GB)

(73) Assignee: KBC ADVANCED TECHNOLOGIES PLC, Surrey (GB)

(21) Appl. No.: 14/378,866

(22) PCT Filed: Feb. 14, 2013
(86) PCT No.: PCT/GB2013/050351

§ 371 Date: Aug. 14, 2014

(30) Foreign Application Priority Data
Feb. 14, 2012 (GB) 1202476.6

(51) Int. Cl. G01N 33/00; G01N 30/00

(52) U.S. Cl. CPC G01N 33/0009; G01N 30/00
USPC 702/24

(57) ABSTRACT

A method of characterizing a mixture having a plurality of hydrocarbons includes: obtaining a set of predetermined pseudo-components, each pseudo-component representing a respective group of hydrocarbons; obtaining physical data about the hydrocarbon mixture; and determining from the data the amount of each pseudo-component considered to be present in the mixture.

The present invention relates to the field of hydrocarbon modelling. In particular, it relates to the field of modelling a mixture of hydrocarbons such as a petroleum fluid in or extracted from an oil well.

Conventionally in the oil industry, thermodynamic or PVT (pressure volume temperature) models of petroleum fluids are set up by first characterising a petroleum fluid by representing it as a set of components.

This process starts by analysing the fluid, typically by gas chromatography (GC). GC is used to detect the often huge number of different hydrocarbons present in petroleum fluids. Typically, the fluid is separated into a gas sample and a liquid sample, which are then analysed using different types of GC column. The GC analysis gives a response, which is taken to indicate component mass, as a function of the retention time, which is taken to indicate the normal boiling point of the components, i.e. a measure of their volatility.

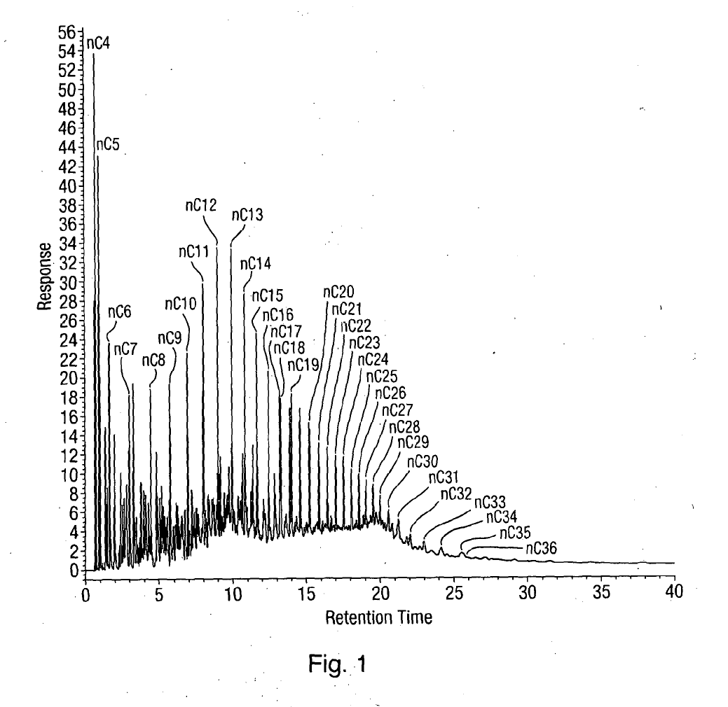

The GC trace generally shows a number of spikes, with each spike corresponding to a n-paraffin (also referred to as a “n-alkane”, with “n” standing for “normal” and meaning a straight chain alkane). The n-paraffins are usually present in relatively large quantities, which is why they appear as spikes in the GC trace. These spikes are used to identify groups of components. FIG. 1 shows an example of a response versus retention time trace for a petroleum fluid.

The response from the GC is integrated in contiguous sections to give a series of single carbon number cuts (SCNs), each SCN including a different n-paraffin spike, and usually running from C6 or C7 up to some upper limit (often C35). Beyond the upper limit there is a remainder of components referred to as the plus fraction, which represents the heavier hydrocarbons in the fluid sample that were not resolved into SCN cuts. Each SCN cut, Cn, represents the n-paraffin with carbon number n plus all those other hydrocarbons that elute between C(n−1) and Cn. The conventional interpretation is that these are the hydrocarbons present in the fluid with normal boiling points lying between n-paraffins C(n−1) and Cn.

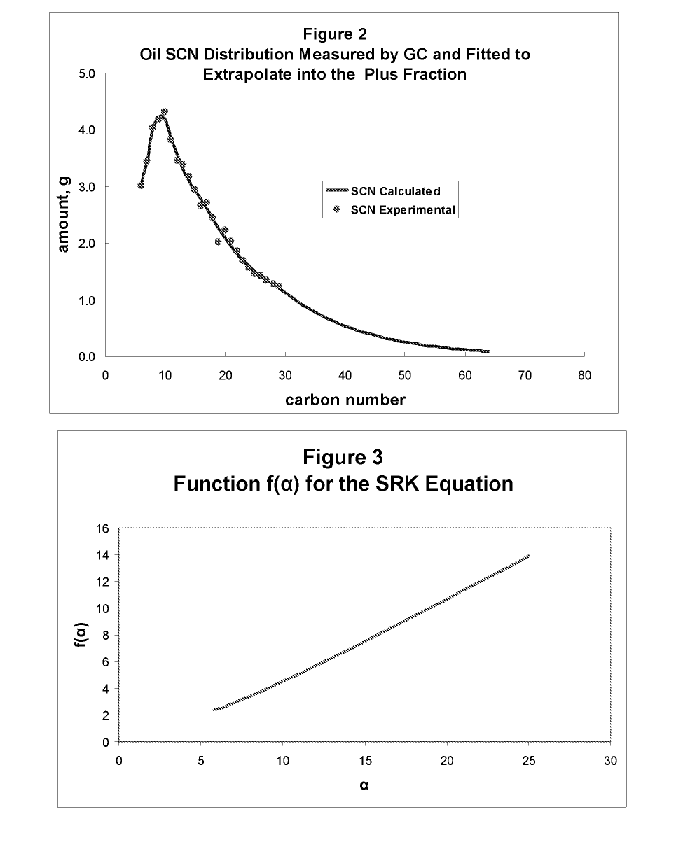

FIG. 2 shows a typical example of a measured SCN distribution obtained by integration of a GC response. In order to estimate what is present in the plus fraction, an empirical function is regressed to fit the data. If information about the molecular weight of the fluid is available, this can be exploited in the regression as molecular weight and carbon number are closely correlated. The function can then be extrapolated to provide an estimate of the SCN distribution in the plus fraction, as illustrated in FIG. 2.

Once a SCN distribution has been determined for the fluid, the properties of the SCN components are estimated so that they can be incorporated in a thermodynamic model, such as an equation of state. Usually, a number of oil industry correlations are employed to achieve this. Many such correlations exist, so what follows is an illustrative example of the methodology.

First, molecular weights, densities and boiling points are assigned to each SCN cut. The correlations of Riazi and Al-Sahhaf (see M. Riazi, T. A. Al-Sahhaf: Fluid Phase Equilibria, 117, 217, 1996), for example, allow these quantities to be estimated from the carbon numbers of each cut. In fact, the normal boiling point is already closely defined as each cut, Cn, represents the n-paraffin of carbon number n plus all the other hydrocarbons that boil between the n-paraffins with carbon numbers n−1 and n. If the molecular weight or density of the fluid has been directly measured, the assigned properties of the cuts can, if necessary, be scaled up or down so as to agree with the measurements.

Although GC analysis is widely used by the upstream oil industry, it is not the only possible method of analysis. Another approach is to distil the liquid sample in a high-reflux column to separate out the constituents by normal boiling point. The end result is a true boiling point (TBP) curve giving the mass of substance distilled off versus the normal boiling point. The TBP curve gives similar information to a GC analysis with the advantage that physical samples can be collected, each one over a measured range of boiling points. The physical properties, such as molecular weight, density and others, can then be directly measured for each sample collected.

Another analytical method is ASTM D86 distillation, which gives a D86 curve. It can also be used as a basis for estimating the SCN distribution of the fluid.

An equation of state requires each component to be characterised in terms of its critical temperature, critical pressure and acentric factor. Many correlations exist for these properties. For example, the Kesler-Lee correlations (see M. G. Kesler, B. I. Lee: Hydrocarbon Processor, 55, 153, March 1976), which are widely regarded as reliable, give expressions for critical temperature and pressure as functions of density and normal boiling point. The Edmister correlation (see W. C. Edmister: Petroleum Refiner, 37, 173, April 1958) can then be used to estimate the acentric factor from the critical temperature and the normal boiling point.

Once the critical conditions and acentric factor of a component are known, it can be used in an equation of state. Equations of state are mathematical expressions that predict the pressure of a fluid as a function of its temperature, volume and composition, for example.

The oil industry tends to use equations of state that are termed cubic equations. The two most widely used are the Soave-Redlich-Kwong (SRK) equation (see G. Soave: Chem. Eng. Sci., 27, 1197, 1972) and the Peng-Robinson (PR) equation (see D.-Y. Peng, D. B. Robinson: Ind. Eng. Chem. Fundam., 15, 59, 1976).

The SRK equation is

$$p = \frac{RT}{v - b} - \frac{a}{v ( v + b )} \qquad ( 1 )$$

while the PR equation is

$$p = \frac{RT}{v - b} - \frac{a}{{v}^{2} + 2 vb - {b}^{2}} \qquad ( 2 )$$

where p is the pressure, T is the absolute temperature, v is the molar volume and R is the gas constant. The parameters a and b describe the properties of the fluid, a being a measure of the molecules' tendency to attract one another and b being a measure of molecular size.

Theoretical analysis shows that for every component at its critical point, the following relations must hold:

$$\begin{matrix}{a}_{c} = {Ω}_{a} \frac{{R}^{2} {T}_{c}^{2}}{{p}_{c}} \\ and & ( 3 ) \\ b = {Ω}_{b} \frac{{RT}_{c}}{{p}_{c}} & ( 4 )\end{matrix}$$

where Ωₐ and Ω_{b} are numerical constants determined by the mathematical form of the equation of state and subscript c denotes a critical value. For the SRK equation Ωₐ=0.42748 and Ω_{b}=0.086640 and for the PR equation Ωₐ=0.45724 and Ω_{b}=0.077796.

In addition, the a parameter has to be treated as a function of temperature:

a=a_{c}(1+κ(1−\sqrt{Tᵣ}))² (5)

where

$${T}_{r} = \frac{T}{{T}_{c}} \qquad ( 6 )$$

and

κ=c₁+c₂ω+c₃ω² (7)

where ω is the acentric factor.

For the SRK equation c₁=0.48, c₂=1.574 and c₃=−0.176 whereas for the PR equation c₁=0.37464, c₂=1.54226 and c₃=−0.26992.

Finally, the equation of state is extended to apply to mixtures of components by introducing mixing rules. Given that the parameters a and b can be found for each individual component present, it is postulated that these parameters can be averaged to determine their values in the mixture.

The oil-industry standard is to use the Van der Waals mixing rules:

$$\begin{matrix}a = {Σ}_{ij} {x}_{i} {x}_{j} \sqrt{{a}_{i} {a}_{j}} ( 1 - {k}_{ij} ) \\ and & ( 8 ) \\ b = {Σ}_{i} {x}_{i} {b}_{i} & ( 9 )\end{matrix}$$

Here, parameters a and b without subscripts denote the mixture average values whereas the subscripted values denote the values of a particular component i or j. xᵢ, xⱼ denote the mole fractions of components i, j in the mixture. kᵢⱼ is the binary interaction parameter (BIP) which is an adjustment factor that can be fitted to binary data for the i,j^{th} component pair. Kᵢⱼ should have a small value and is typically zero for many hydrocarbon pairs.

The Van der Waals mixing rules are empirical expressions that are largely chosen for their mathematical convenience. However, experience shows that they only satisfactorily represent the properties of mixtures of non-polar or mildly polar molecules, and only when those molecules are fairly similar in size. With some use of BIPs, the mixing rules can be employed to represent petroleum fluids; the limitations are not too apparent as the oil industry normally focuses on predictions that are not too severely compromised.

However, representing petroleum fluids as sets of SCN cuts requires a relatively large number of components, the properties of many of which are quite similar. It is therefore usual to group the SCN cuts into a smaller number of so-called pseudo-components, each pseudo-component representing a range of SCN cuts from a lower to an upper carbon number. For example, a SCN distribution measured by GC up to C36 might be cut into pseudo-components for C6-C9, C10-C15, C16-C22, C23-C33 and C34+.

The properties of each pseudo-component are then determined from its constituent SCN cuts using some averaging rules with the object of making the contributions of the pseudo-components to parameters a and b about the same as that of the constituent SCN cuts. The number of pseudo-components required depends of the desired detail of the equation-of-state predictions. For many applications, about five pseudo-components gives good results.

Once the equation-of-state model has been set up, it is possible to compute all the PVT properties of the fluid. However, because the compositions of petroleum fluids are so variable, it is nearly always the case that the equation-of-state predictions are inconsistent with the actual measured PVT properties of the oil in question. The normal approach is then to adjust the critical properties or acentric factors of the pseudo-components to match the measured properties of the fluid, such as the saturation line or separator test data. The fit to the measured volumetric properties can also be improved by altering the pseudo-component properties, although a better approach is often to introduce volume shifts following the method of Peneloux (see A. Peneloux, E. Rauzy, R. Freze: Fluid Phase Equilibria, 8, 7, 1982).

With this normal oil-industry methodology, every petroleum fluid is represented by its own set of unique pseudo-components and the properties assigned to the pseudo-components of each fluid are different. For studies involving single fluids this presents little difficulty but nearly all practical engineering studies involve several fluids.

For example, when an oil field is appraised, many samples are typically taken from different wells or from the same well at different times. Such samples will all be different to a greater or lesser degree depending on measurement accuracy and where in the reservoir they are taken from.

In addition, situations involving commingled production are becoming increasingly common. Examples include production facilities for fields with fluids of varying properties often through multilateral wells, tie-backs of new fields to existing facilities, pipeline networks linking multiple fields and refineries that use many feed-stocks.

Design studies of commingled production of many different fluids can lead to an unacceptable number of pseudo-components. The number of components used in a complex simulation usually needs to be kept down, which often leads to a requirement that a common set of pseudo-components is used to model all the fluids involved. This requirement can cause petroleum engineers to expend large amounts of time in order to find a suitable set of common pseudo-components.

The underlying problem is that the current methodology depends on varying the pseudo-component properties of each fluid to represent its properties. This means that finding a common set of pseudo-components that will simultaneously represent the properties of several fluids is incompatible with the basis of the conventional methodology. Often, the properties of common pseudo-components have to be a compromise that represents all of the individual fluids less well than their own dedicated pseudo-components would.

The inventors of the present invention realised that a quick and reliable method to represent multiple fluids with one common set of pseudo-components could improve the accuracy of equation-of-state simulations for engineering design studies and also save large amounts of engineers' time.

Thus, the present invention seeks to provide a method for representing multiple fluids by a common set of pseudo-components in order to provide greater convenience, improved speed and accuracy of the calculations and savings in engineers' time.

According to a first aspect of the present invention, there is provided a method of characterising a mixture comprising a plurality of hydrocarbons, said method comprising: providing a set of predetermined pseudo-components, each pseudo-component representing a respective group of hydrocarbons; obtaining physical data about the hydrocarbon mixture; and determining from the data the amount of each pseudo-component considered to be present in the mixture.

According to a second aspect, there is provided a computer program for characterising a mixture comprising a plurality of hydrocarbons, said computer program being configured to perform the following steps when executed on a computer: obtain a set of predetermined pseudo-components, each pseudo-component representing a respective group of hydrocarbons; obtain physical data about the hydrocarbon mixture; and determine from the data the amount of each pseudo-component present in the mixture.

According to a third aspect, there is provided a computer readable medium with a computer program as described above, optionally with any of the further features described below, stored thereon.

According to a fourth aspect, there is provided a computer system configured to perform the method described above, optionally with any of the further features described below.

According to the aspects described above, the present invention thereby allows mixtures of hydrocarbons to be characterised in terms of the amounts of various predetermined pseudo-components that those mixtures can be considered to contain.

Each pseudo-component represents a group of hydrocarbons. For example, a pseudo-component could represent the hydrocarbons with boiling points lying within a section (i.e. between two points on an x-axis) of a GC trace, or a trace obtained from a similar analytical method such as those mentioned in this application.

The predetermined pseudo-components have predetermined physical properties which can be used to characterise the hydrocarbon mixture. The predetermined physical properties may include the critical temperature, critical pressure and/or acentric factor, for example.

The step of obtaining physical data about the hydrocarbon mixture could simply involve obtaining that data from a computer memory or other data storage component, for example, where it is has been previously stored. It could additionally or alternatively involve performing an analysis (e.g. GC analysis) of the hydrocarbon mixture in order to obtain the data.

The set of predetermined pseudo-components preferably comprises a distribution of pseudo-components according to hydrocarbon carbon number. For example, the set of predetermined pseudo-components may comprise a distribution of single carbon number cuts (SCNs). Alternatively, the set of predetermined pseudo-components may comprise a distribution of SCNs grouped together into pseudo-components comprising more than one SCN with averaged properties. This would reduce the number of pseudo-components required, thereby reducing computing time. The SCNs may be grouped together into 3-10 pseudo-components, preferably 4-7 pseudo-components, and more preferably 5 pseudo-components.

Ideally, two or more such distributions of predetermined of pseudo-components are used. For example, two distributions of predetermined pseudo-components representing the aromatic and paraffinic constituents of the mixture, respectively, could be used. Although this method requires twice as many components to represent any one fluid (i.e. paraffinic and aromatic pseudo-components for each SCN cut or group of cuts), as soon as more than one fluid is involved, the benefit is that no more pseudo-components are required.

In some applications of the invention, such as those described below, it might be preferred to have more than two distributions of pseudo-components, depending on which groups of molecules in the mixture are of particular interest. For example, in order to model wax precipitation three distributions of predetermined pseudo-components could be used representing the n-paraffins, the aliphatic components (i.e. the non-aromatic part of the mixture, without the n-paraffins) and the aromatic components, respectively. Another example is modelling an asphaltenic oil by characterising it as a mixture of pseudo-component distributions representing the paraffinic, naphthenic, aromatic, resinous and asphaltenic fractions of the oil.

Provided that the boundaries between pseudo-components are set the same for all the fluids in a particular simulation, the SCN components for each fluid may be grouped into fewer, coarser pseudo-components and the method can be applied with a reduced number of predetermined pseudo-components. For example, if a pseudo-component is defined as containing all SCN cuts from Cm to Cn, a Cm-Cn aromatic generic pseudo-component is defined and a Cm-Cn paraffinic generic pseudo-component is defined. Each such generic pseudo-component is assigned fixed physical properties. These two generic pseudo-components are then used to represent all Cm to Cn cuts in all fluids being simulated. Likewise, other pairs of generic pseudo-components are used to represent the other carbon number ranges of cuts.

Preferably, the physical data is analytical data provided, for example, from a gas chromatography analysis of the hydrocarbon mixture. Alternatively, methods such as TBP or ASTM D86 distillation, or similar, could be used to obtain analytical data.

The amount of each predetermined pseudo-component present in the mixture may be determined by adjusting blend ratios of the (e.g. aromatic and paraffinic) pseudo-components until they reproduce the physical properties of the mixture. The correct overall concentrations of each carbon number cut should be preserved while this step is performed.

The properties of the mixture may be predicted by averaging the properties of the constituent pseudo-components.

The properties of the mixture may be modelled using an equation of state, for example a cubic equation of state such as the SRK or PR equation given above.

The modelling preferably comprises using a mixing rule for the equation of state which is ideally adapted for an athermal and/or an ideal solution. The mixing rule allows equation of state constants, such as a and b in the SRK and PR equations, to be estimated by combining the contributions from all of the pseudo-components in the mixture. The mixing rule for b could be the standard mixing rule given by equation 9 and the mixing rule for a could be adapted for an ideal or athermal solution.

Preferably, the mixing rule is adapted for an athermal and/or an ideal solution.

The mixing rule could be:

(i) adapted for an ideal solution;
(ii) adapted for an athermal solution; or
(iii) adapted for an ideal and an athermal solution, for example by including contributions according to both of the above mixing rules (i) and (ii).

For most hydrocarbon mixtures, athermal is probably the best approximation to describe their behaviour so a mixing rule adapted entirely for an athermal solution would be appropriate.

However, a large number of mixtures behave somewhere between an ideal solution and an athermal solution. In this case, the mixing rule could be adapted for both an athermal and an ideal solution. For example, it could be a combination of the mixing rules adapted for athermal and ideal solutions. This combination could be formed by including a switching function to include contributions according to either the athermal or the ideal mixing rule, depending on which rule is most appropriate for a particular fluid.

The mixing rule could be adapted for an ideal or athermal solution based on thermodynamic properties of such solutions. For example, the mixing rule could be adapted for an ideal or athermal solution based on the thermodynamic definition of the Gibbs energy or the enthalpy of such a solution. For an ideal solution, the excess Gibbs energy is zero and for an athermal solution the excess enthalpy is zero so these are convenient parameters to use.

In the case that the mixing rule is adapted for an athermal solution, it is preferably given by:

$$g ( α ) = {\sum }_{i} {x}_{i} g ( {α}_{i} )$$

where xᵢ is the mole fraction of component i in the mixture; and the method comprises solving the mixing rule numerically to find α and then calculating a for the mixture from a=RTbα, where R is the universal gas constant, T is the absolute temperature, and b is determined from

$$b = {\sum }_{i} {x}_{i} {b}_{i} .$$

In the case that the mixing rule is adapted for an ideal solution, it is preferably given by:

$$f ( α ) = {\sum }_{i} ( {x}_{i} ( f ( {α}_{i} ) + ln {b}_{i} ) ) - ln b$$

where xᵢ is the mole fraction of component i in the mixture, b is a constant in an equation of state determined from

$$b = {\sum }_{i} {x}_{i} {b}_{i} ,$$

and the method comprises solving the mixing rule numerically to find α and then calculating a for the mixture from a=RTbα.

The derivation of these mixing rules from thermodynamic principles is discussed in more detail below.

In order to apply the mixing rules to the pseudo-components, the values of the equation of state constants, e.g. a and b, are ideally first estimated for each pseudo-component. This can be performed on the basis of that pseudo-component's critical temperature and critical pressure (using equations 3-7), for example. The values of the constants for the mixture may then be estimated using the mixing rules for the constants.

The mixing rule may additionally or alternatively be adapted for polar components, for example with binary interaction parameters. This is described in more detail below.

The mixing rule may additionally or alternatively be adapted to account for any contribution from gases, e.g. hydrocarbon or other gases. This is described in more detail below.

The method may further involve simulating blending a plurality of mixtures together, each mixture having been characterised using the method described above, by adding molar or mass flow rates for each pseudo-component, for example. The plurality of mixtures should ideally all be characterised using the same pseudo-components. This makes simulating combining the mixtures together particularly simple.

Each pseudo-component may be regarded as a generic pseudo-component, i.e. one which can be used to characterise a plurality of hydrocarbon mixtures. For example, a first mixture may be determined to have a first amount of a generic pseudo-component present therein, and a second mixture may be determined to have a second amount of that generic pseudo-component present therein. If the mixtures are blended, for example, then the contribution to the resulting blended mixture may be determined based on the first and second amounts of the generic pseudo-components considered to be present in the resulting blended mixture.

The mixture may be a petroleum fluid, for example. However, the method may be used for other hydrocarbon mixtures.

The method described above could be applied to studies of any petroleum fluids and their commingling. For example, it could be used in subsurface studies, pipeline network studies and processing studies in general. It could also be used in various specific applications such as modelling wax precipitation in a fluid, modelling contamination from drilling mud in a petroleum sample, determining allocation agreements for a group of oil wells, and modelling polymers, solubility, asphaltenes and/or naphthenic acids.

The inventors have realised that the idea of using two or more distributions of predetermined of pseudo-components, each representing a different group of components, to characterise a mixture is of independent inventive significance, regardless of whether or not the pseudo-components are predetermined.

Thus, viewed from a fifth aspect, there is provided a method of characterising a mixture comprising a plurality of hydrocarbons, the method comprising: obtaining physical data about the mixture; defining two or more distributions of pseudo-components, each distribution representing a respective group of hydrocarbons or other constituent of the mixture, and each pseudo-component of each distribution representing a respective sub-group of that distribution; determining from the data the amount of each pseudo-component of each distribution considered to be present in the mixture; and applying a mixing rule to the pseudo-components, wherein the mixing rule is adapted for an athermal and/or an ideal solution.

According to a sixth aspect, there is provided a computer program for characterising a mixture comprising a plurality of hydrocarbons, said computer program being configured to perform the following steps when executed on a computer: obtain physical data about the mixture; define two or more distributions of pseudo-components, each distribution representing a respective group of hydrocarbons or other constituent of the mixture, and each pseudo-component of each distribution representing a respective sub-group of that distribution; determine from the data the amount of each pseudo-component of each distribution considered to be present in the mixture; and apply a mixing rule to the pseudo-components, wherein the mixing rule is adapted for an athermal and/or an ideal solution.

According to a seventh aspect, there is provided a computer readable medium with a computer program as described above in relation to the sixth aspect, optionally with any of the further features described above or below, stored thereon.

According to an eighth aspect, there is provided a computer system configured to perform the method described above in relation to the fifth aspect, optionally with any of the further features described above or below.

According to the fifth to eighth aspects of the invention, a mixture comprising a plurality of hydrocarbons may be characterised using two or more distributions of pseudo-components, each distribution representing a different group of constituents of the mixture. Each distribution preferably represents a group of constituents of the mixture with similar properties, particularly similar chemical and/or physical properties. For example, a mixture could be characterised by aromatic and paraffinic distributions. This means that studies can be performed where different groups of constituents, each group having similar properties, can be treated separately.

Although using more than one distribution has been contemplated in the past, mixtures characterised in this way were still subjected to the Van der Waals mixing rules before performing an equation of state simulation. However, the Van der Waals mixing rules do not work because they give incorrect results when more than one distribution of pseudo-components is used. Therefore, simulations based on such characterisations do not produce useful or reliable results.

In contrast with this, the inventors of the present invention realised that by using mixing rules adapted to reflect athermal and/or ideal solutions, this would produce reliable results when performing an equation of state simulation based on the results of such mixing rules, when applied to mixtures characterised according to two or more distributions of pseudo-components.

Therefore, advantages of characterising a mixture according to two or more distributions of pseudo-components can be obtained if athermal or ideal solution mixing rules are applied, rather than the conventional Van der Waals mixing rules.

As explained above, the mixing rules allow equation of state constants, such as a and b from the SRK and PR equations, to be estimated by combining the contributions from each pseudo-component. By ‘applying a mixing rule to the pseudo-components’ is meant applying a mixing rule to properties of the pseudo-components, in particular the pseudo-components' values for equation of state constants a and b.

The mixing rule could be:

(i) adapted for an ideal solution;
(ii) adapted for an athermal solution; or
(iii) adapted for an ideal and an athermal solution, for example by including contributions according to both of the above mixing rules (i) and (ii).

As explained above, for most hydrocarbon mixtures, athermal is probably the best approximation to describe their behaviour so a mixing rule adapted entirely for an athermal solution would be appropriate.

However, a large number of mixtures behave somewhere between an ideal solution and an athermal solution. In this case, the mixing rule could be adapted for both an athermal and an ideal solution. For example, it could be a combination of the mixing rules adapted for athermal and ideal solutions. This combination could be formed by including a switching function to include contributions according to either the athermal or the ideal mixing rule, depending on which rule is most appropriate for a particular fluid.

Preferably, the mixing rule is adapted for an athermal and/or an ideal solution based on thermodynamic properties of such a solution. For example, the mixing rule could be adapted for an ideal or athermal solution based on the thermodynamic definition of the Gibbs energy or the enthalpy of such a solution. For an ideal solution, the excess Gibbs energy is zero and for an athermal solution the excess enthalpy is zero so these are convenient parameters to use.

In the case that the mixing rule is adapted for an athermal solution, it is preferably given by:

$$g ( α ) = {\sum }_{i} {x}_{i} g ( {α}_{i} )$$

where xᵢ is the mole fraction of component i in the mixture; and the method comprises solving the mixing rule numerically to find α and then calculating a for the mixture from a=RTbα, where R is the universal gas constant, T is the absolute temperature, and b is determined from

$$b = {\sum }_{i} {x}_{i} {b}_{i} .$$

In the case that the mixing rule is adapted for an ideal solution, it is preferably given by:

$$f ( α ) = {\sum }_{i} ( {x}_{i} ( f ( {α}_{i} ) + ln {b}_{i} ) ) - ln b$$

where xᵢ is the mole fraction of component i in the mixture, b is a constant in an equation of state determined from

$$b = {\sum }_{i} {x}_{i} {b}_{i} ,$$

and the method comprises solving the mixing rule numerically to find α and then calculating a for the mixture from a=RTbα.

The derivation of these mixing rules from thermodynamic principles is discussed in more detail below.

In order to apply the mixing rules to the pseudo-components, the values of the equation of state constants, e.g. a and b, are ideally first estimated for each pseudo-component. Such constants may be estimated on the basis of that pseudo-component's critical temperature and critical pressure (using equations 3-7), for example. The values of the constants for the mixture may then be estimated using the mixing rules for the constants.

The method may further comprise then modelling properties of the mixture using an equation of state.

In the case where two pseudo-component distributions are used, the two distributions preferably represent the aromatic and paraffinic components of the mixture respectively. These two groups of pseudo-components represent two ‘extremes’, in terms of chemical properties, of hydrocarbons so can be a good way to characterise a mixture.

The method may comprise modelling wax precipitation in the mixture. In this case, one of the two or more pseudo-component distributions ideally represents the n-paraffins present in the mixture. This is because these are the components that crystallise into a wax. Preferably, for wax modelling studies, three pseudo-component distributions are used, the three distributions representing aromatic, n-paraffin, and aliphatic components of the mixture respectively.

The method may comprise modelling polymers.

The mixing rule may additionally be adapted for polar components, for example with binary interaction parameters. This is discussed in more detail below.

The mixing rule may additionally be adapted to account for any contribution from gases, e.g. hydrocarbon or other gases. This is discussed in more detail below.

The fifth to eighth aspects of the invention may include any of the features (including the optional or preferred features) of the first to fourth aspects of the invention described above.

Preferred embodiments of the invention will now be described by way of example only and with reference to the accompanying figures in which:

FIG. 1 shows an example of a GC response versus retention time trace for a petroleum fluid;

FIG. 2 is a typical example of a measured SCN distribution;

FIG. 3 shows the function ƒ(α) for the SRK equation plotted versus α;

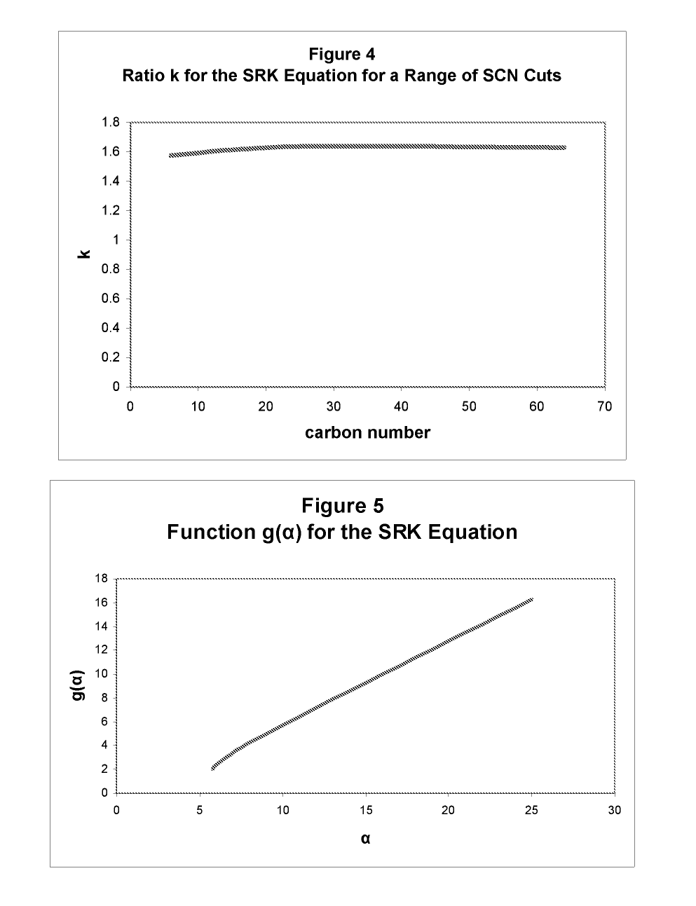

FIG. 4 shows the ratio k using the Riazi-Al-Sahhaf, Kesler-Lee and Edmister correlations over a range of carbon numbers at a fixed temperature;

FIG. 5 shows the function g(α) for the SRK equation plotted versus α; and

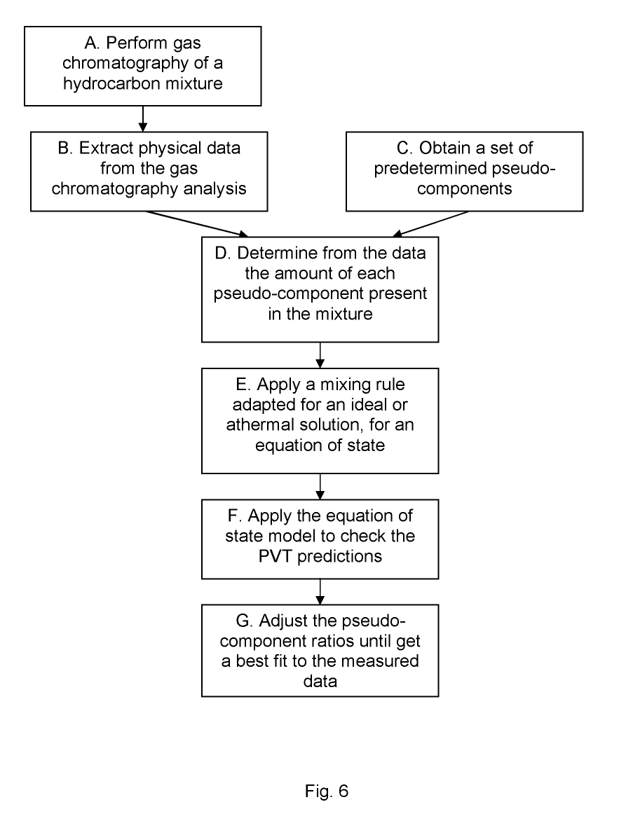

FIG. 6 is a flow chart illustrating an embodiment of the method.

The present invention can be used to model a hydrocarbon mixture, such as a petroleum fluid, or a group of such mixtures in the following way.

First, a SCN distribution is set up from analytical data (e.g. GC data) for a hydrocarbon mixture in the conventional way. The molecular weights, densities and normal boiling points are then estimated using correlations such as those of Riazi and Al-Sahhaf. Alternatively, TBP distillation or a similar analytical method could be employed.

In order to set up a predetermined set of pseudo-components, initially one hydrocarbon mixture is selected, preferably the heaviest if taken from a study of a plurality of mixtures. The reason for selecting the heaviest mixture is to ensure that the SCN distribution contains contributions at the higher end of the SCN scale, as well as the lower end.

Each of the mixture's SCN cuts is represented as a blend of the aromatic and paraffinic SCN cut of the same carbon number. The blending ratio of the aromatic and paraffinic cuts is adjusted to represent the actual properties of the measured cut.

Usually, the SCN cuts are grouped into a smaller number of pseudo-components. So here, the aromatic and paraffinic cuts are grouped into generic aromatic and paraffinic pseudo-components between defined carbon number boundaries that are appropriate for the fluid and/or study in question, e.g. C6-C9, C10-C15, C16-C22, C23-C33 and C34+.

How the SCN components are grouped into pseudo-components (i.e. where the boundaries lie and how many pseudo-components to use) will depend on how much detail a particular study requires. For example, in a study looking at how a petroleum oil seeps into rocks, it might be sufficient to have a single aromatic and a single paraffinic pseudo-component to represent the whole mixture because there are many other factors that will affect this situation besides the composition of the oil. In contrast, when performing oil pipeline studies, it will usually be better to have more pseudo-components, for example five in each distribution (i.e. aromatic and paraffinic). Other studies might benefit from having even more pseudo-components.

In addition, when deciding where to put the boundaries between pseudo-components, there will usually be a compromise between the conflicting aims of ensuring that each pseudo-component represents approximately the same mass or proportion of the mixture, and having cuts with the same width so that they each contain the same spread of volatilities.

The physical properties (e.g. critical temperature, critical pressure, acentric factor) of the generic pseudo-components are found from the properties of their constituent SCN cuts using a suitable averaging rule. The physical properties assigned to these pseudo-components are fixed and these pseudo-components form predetermined pseudo-components that can then be used to characterise all the other mixtures in the study, or even in further studies.

In order to characterise any further mixture of hydrocarbons, the method of FIG. 6 is used. Here, a SCN distribution is obtained for the further mixture (step A) from, for example, GC or TBP distillation analysis, and physical data is obtained by analysing the SCN distribution (step B).

A predetermined set of pseudo-components (such as that described above) is obtained, e.g. from a memory in a computer, (step C) and the SCN cuts of the mixture that is being characterised are then grouped into pseudo-components using the same carbon number boundaries as for the predetermined pseudo-components.

Each pseudo-component of the further mixture can then be represented as a blend of the corresponding aromatic and paraffinic pseudo-components. For each pseudo-component, the blend ratio of aromatic to paraffinic pseudo-components is adjusted to reproduce the physical properties of the pseudo-component in question; these properties can be deduced by averaging the properties of the constituent SCN cuts (step D).

At this point, any mixture can be described by a mixture of the predetermined aromatic and paraffinic pseudo-components. Each mixture may differ in the relative amounts of the pseudo-components from which it is formed but the properties of the pseudo-components are fixed and common to all the mixtures. Further mixtures can be characterised using the same set of predetermined pseudo-components by following the same method.

In order to model the properties of the mixtures, an equation of state with critical properties and acentric factors assigned to each pseudo-component according to standard correlations, such as the Kesler-Lee and Edmister expressions, is used. However, in order to obtain reliable predictions, the equation of state is used in combination with a mixing rule that will correctly describe the thermodynamic properties of a hydrocarbon mixture. Therefore, mixing rules adapted for athermal solutions, or something similar such as a mixing rule adapted for ideal solutions, are used (step E), but not the conventionally used Van der Waals mixing rules, which fail if more than one generic distribution of pseudo-components is used. As explained below, only the mixing rule for equation of state constant a need be adapted for an ideal or athermal solution. The standard mixing rule for b can still be used (equation 9).

The equation-of-state model is then applied to check the predictions of the PVT properties of each mixture (step F). The model for each mixture may be adjusted by altering the aromatic-paraffinic pseudo-component ratios until a best fit is obtained to the measured data (step G). The model is then ready for use.

Any operation in which different fluids or mixtures are blended can be represented very simply by adding the molar or mass flow-rates of each generic pseudo-component together to give the corresponding flow-rates in the commingled fluid; no new pseudo-components are required.

There is no theoretical reason to suppose that the Van der Waals mixing rules used in cubic equations of state accurately describe the thermodynamic properties of real mixtures. Experience suggests that when the components in the mixture do not differ much in molecular size, i.e. when their equation-of-state parameters have similar magnitudes, the predicted thermodynamic behaviour is reasonable for hydrocarbons. However, as the size ratio of the equation-of-state parameters increases, the predictions become increasingly unrealistic.

It is known that, at moderate pressures, hydrocarbons form an approximately ideal solution, i.e. one where the excess Gibbs energy (GE) is zero. When petroleum fluids are represented by a distribution of pseudo-components, it is found that the mixture is reasonably represented by a cubic equation of state provided that there is only one distribution of pseudo-components present. In this case, the pseudo-components are predicted to form a mixture that is not too different from an ideal solution. However, if more than one distribution of pseudo-components is present, the pseudo-components can show very high deviations from ideal, often leading to an erroneous prediction that the fluid will separate into two coexisting liquid phases.

An example where one might wish to use more than one distribution would be to perform waxing calculations where the distribution of the n-paraffins (or n-alkanes) must be separated off from the other components, because waxes are crystals formed predominantly of n-paraffins.

Due to of the erroneous predictions of the conventional Van de Waals mixing rules, it is not possible to model the thermodynamics of petroleum fluids using two or more distributions of pseudo-components using these mixing rules. Consequently, a method involving aromatic and paraffinic distributions of pseudo-components, for example, will not work sufficiently accurately with the conventional mixing rules.

Improvements or modifications must be made to the mixing rules so that the properties of hydrocarbon mixtures can be correctly reproduced.

In order to model mixtures of hydrocarbons accurately, it is better to use mixing rules that reflect the known thermodynamic behaviour of the mixture in question.

Taking the SRK equation (equation 1), and using the thermodynamic relationship p=−∂A/∂v, the Helmholtz energy A is found to be

$$A = - RT ln ( v - b ) - \frac{a}{b} ln ( \frac{v + b}{v} ) \qquad ( 10 )$$

Here, A is the molar Helmholtz energy and v is the molar volume. The other nomenclature is as before. Hence, from its fundamental definition, the molar Gibbs energy G is

$$G = - RT ln ( v - b ) - \frac{a}{b} ln ( \frac{v + b}{v} ) + pv \qquad ( 11 )$$

Since the thermodynamic properties of liquids are only slightly affected by pressure, it is mathematically convenient to set the pressure to zero in the expression for the Gibbs energy (equation 11) as that gives its lower limiting value. Defining u=v₀/b where v₀ is liquid molar volume at zero pressure, and defining α=a/RTb, it follows for the SRK equation (equation 1) that

$$\begin{matrix}\frac{RT}{{v}_{0} - b} - \frac{a}{{v}_{0} ( {v}_{0} + b )} = 0 \\ or & ( 12 ) \\ \frac{u ( u + 1 )}{u - 1} = α & ( 13 )\end{matrix}$$

which, selecting the physically meaningful root, gives an explicit expression for u of

$$u = \frac{1}{2} ( α - 1 - \sqrt{{α}^{2} - 6 α + 1} ) \qquad ( 14 )$$

The molar Gibbs energy at zero pressure can therefore be expressed as

$$\begin{matrix}\frac{G}{RT} = - ln ( u - 1 ) - αln ( \frac{u + 1}{u} ) - ln b \\ or & ( 15 ) \\ \frac{G}{RT} = - f ( α ) - ln b & ( 16 )\end{matrix}$$

Since u is a function of α, ƒ(α) is also a pure function of α

$$f ( α ) = ln ( u - 1 ) + α ln ( \frac{u + 1}{u} ) \qquad ( 17 )$$

FIG. 3 shows the function ƒ(α) for the SRK equation (equation 1) plotted versus α.

An excess function is defined as the value of that function for a liquid mixture minus the sum of the values of the same function for each of the mixture components if present as a pure liquid at the same temperature and pressure. Thus, for the SRK equation (equation 1) the excess liquid molar Gibbs energy is

$$\frac{{G}^{E}}{RT} = - ( f ( α ) + ln b ) + {\sum }_{i} ( {x}_{i} ( f ( {α}_{i} ) + ln {b}_{i} ) ) \qquad ( 18 )$$

where subscripts i denote the values for the pure components.

For an ideal solution where the excess Gibbs energy is zero, the function ƒ for the mixture must be

$$f ( α ) = {\sum }_{i}^{ } ( {x}_{i} ( f ( {α}_{i} ) + ln {b}_{i} ) ) - ln b \qquad ( 19 )$$

We can, for example, continue to use the conventional mixing rule for b (equation 9), which enables us to obtain ƒ(α) for the mixture.

Equation 19 can then be implicitly solved to obtain α using a suitable numerical method.

The time taken to solve equation 19 is independent of the number of components in the mixture and is not particularly significant in proportion to the time needed to process the equation of state as a whole.

The mixture value of the a parameter then follows from the definition of α: a=RTbα.

Using this procedure to obtain a ensures that the equation of state predicts that the solution at zero pressure is exactly ideal, and at moderate pressures it will remain nearly ideal.

In fact, it is known that hydrocarbon mixtures do not exactly form an ideal solution. A slightly more accurate description of these mixtures is that they form an athermal solution (see J. A. P. Coutinho, S. I. Andersen, E. H. Stenby: Fluid Phase Equilibria, 103, 23, 1995), i.e. one where the excess enthalpy is zero.

A variation of the procedure above can easily be defined to model an athermal solution.

Using the thermodynamic relationship for the internal energy U

$$U = - {RT}^{2} \frac{\partial }{\partial T} ( \frac{A}{RT} ) \qquad ( 20 )$$

the molar internal energy for the SRK equation (equation 1) becomes

$$U = - \frac{1}{b} ( a - T \frac{\partial a}{\partial T} ) ln ( \frac{v + b}{v} ) \qquad ( 21 )$$

From its fundamental thermodynamic definition, the molar enthalpy His therefore

$$H = - \frac{1}{b} ( a - T \frac{\partial a}{\partial T} ) ln ( \frac{v + b}{v} ) + pv \qquad ( 22 )$$

At this point it is necessary to introduce an approximation because temperature derivatives of the parameter a need to be eliminated in order to obtain a procedure for defining the mixture values of a.

If we calculate the critical properties and acentric factors of petroleum fractions using industry-standard procedures, we can obtain values of a and ∂a/∂T as a function of molecular weight. Using the Riazi-Al-Sahhaf, Kesler-Lee and Edmister correlations for example, we show in FIG. 4 the values of the ratio k

$$k = \frac{( a - T \frac{\partial a}{\partial T} )}{a} \qquad ( 23 )$$

over a very wide range of carbon numbers at a fixed temperature.

It can be seen that the value of k only varies by a few percent. We can therefore make the approximation that k is approximately constant at a given temperature.

Hence the molar enthalpy H simplifies to

$$H = - \frac{ka}{b} ln ( \frac{v + b}{v} ) + pv \qquad ( 24 )$$

At zero pressure the enthalpy can be written as

$$\begin{matrix}\frac{H}{RT} = - k α ln ( \frac{u + 1}{u} ) \\ or & ( 25 ) \\ \frac{H}{RT} = - kg ( α ) & ( 26 )\end{matrix}$$

where g(α) is a pure function of α given by

$$g ( α ) = α ln ( \frac{u + 1}{u} ) \qquad ( 27 )$$

FIG. 5 shows the function g(α) for the SRK equation (equation 1) plotted versus α.

The excess liquid molar enthalpy is consequently given by

$$\frac{{H}^{E}}{RT} = - kg ( α ) + {\sum }_{i}^{ } {x}_{i} kg ( {α}_{i} ) \qquad ( 28 )$$

For an athermal solution, where the excess enthalpy is zero, the function g for the mixture must be

$$g ( α ) = {\sum }_{i}^{ } {x}_{i} g ( {α}_{i} ) \qquad ( 29 )$$

Equation 27 can then be implicitly solved to obtain α using a suitable numerical method.

The mixture value of the a parameter then follows from the definition of α: a=RTbα.

Using this procedure to obtain a ensures that the equation of state predicts that the solution at zero pressure is nearly athermal, depending on the accuracy of the assumption that k is constant; at moderate pressures it will remain nearly athermal.

In practice, virtually all modelling problems of petroleum fluids involve mixtures that contain hydrocarbon and other gases. The methods described above are based on excess thermodynamic quantities that describe the effect of mixing pure liquids to form a mixture. The mathematical formalism breaks down at the point where the equation of state must be solved at zero pressure to obtain v₀. If a gas is at a sufficiently high temperature, equation 12 has no real solution because the predicted pressure from equation 1 is positive at all finite molar volumes. As a consequence, the function ƒ(α) cannot be evaluated at low values of α.

In order to eliminate the problem, the function ƒ(α) must be modified.

Below a certain value α=α₀, ƒ(α) is set equal to h(α), a polynomial in α, or some other simple function of a. In order to preserve smoothness of the thermodynamic properties, h(α) is splined on to ƒ(α) at α=α₀, i.e. the coefficients of h(α) are set so that h and its first and second derivatives with respect to a are equal to ƒ and its first and second derivatives with respect to α at α=α₀. The mathematical form of the function h(α) and the value of α₀ are empirically chosen so that the solubility of light hydrocarbon gases in liquid hydrocarbons is correctly reproduced by the model.

An analogous procedure must be followed for function g(α).

In practice, petroleum fluids will contain other components besides hydrocarbons. For example, some gases such as carbon dioxide, hydrogen sulphide, or others may be present. Water may be present and also some oilfield chemicals like methanol or glycols. These polar components will interact in ways that will deviate significantly from ideal or athermal behaviour. In order to model these components, the mixing rules need to be modified to include binary interaction parameters.

Equation 29 may, for example, be generalised to

$$g ( α ) = \frac{1}{2} {\sum }_{ij}^{ } {x}_{i} {x}_{j} ( g ( {α}_{i} ) + g ( {α}_{j} ) ) ( 1 - {k}_{ij} ) \qquad ( 30 )$$

where kᵢⱼ is the binary interaction parameter (BIP) between components i and j.

If all kᵢⱼ are set to zero, equation 30 reverts to the previous expression, equation 29.

Other expressions could be used for equation 30 provided they also have the property of reducing to equation 29 when the BIPs are zero.

Using equation 30 allows kᵢⱼ to be set to zero for pairs of hydrocarbons but adjusted to a suitable non-zero value for pairs of components where one or more are polar.

Likewise function ƒ(α) in equation 19 can, for example, be generalised to

$$f ( α ) = \frac{1}{2} {\sum }_{ij}^{ } {x}_{i} {x}_{j} ( f ( {α}_{i} ) + f ( {α}_{j} ) ) ( 1 - {k}_{ij} ) + {\sum }_{i}^{ } {x}_{i} ln {b}_{i} - ln b \qquad ( 31 )$$

The proposed mixing rules above have been illustrated with the SRK equation of state (equation 1). However, the same principle can be applied with any cubic equation of state or in many models that are based on cubic equations of state such as the cubic plus association (CPA) model.

The PR equation (equation 2) is very widely used and the analysis above can equally well be used with the PR equation and its variants to give analogous results. The expressions for the Helmholtz energy, Gibbs energy, internal energy and enthalpy are all slightly different from those for the SRK equation. In particular equation 14 becomes

$$u = \frac{1}{2} ( α - 2 - \sqrt{{α}^{2} - 8 α + 8} ) \qquad ( 32 )$$

equation 17 becomes

$$f ( α ) = ln ( u - 1 ) + \frac{α}{2 \sqrt{2}} ln ( \frac{u + 1 + \sqrt{2}}{u + 1 - \sqrt{2}} ) \qquad ( 33 )$$

and equation 27 becomes

$$g ( α ) = \frac{α}{2 \sqrt{2}} ln ( \frac{u + 1 + \sqrt{2}}{u + 1 - \sqrt{2}} ) \qquad ( 34 )$$

However, the other relations used for the mixing rules to determine a remain the same as for the SRK equation.

As mentioned above, the present invention has many practical applications. A few examples are described below.

Petroleum wax is a crystalline precipitate that occurs in oils and condensates when the temperature drops below the wax appearance temperature. Wax crystals are solid solutions predominantly composed of n-paraffins.

A number of thermodynamic models exist for wax precipitation but they require knowing the concentration of n-paraffins in the petroleum fluid and also a correct description of the solution of n-paraffins in the fluid.

Investigations of oil-wax equilibria show that the n-paraffins in oil form an approximately ideal solution.

Accurate measurements of binary mixtures of heavy hydrocarbons show in fact a slightly negative deviation from ideal, which would be consistent with the assumption of an athermal solution.

Present methods for modelling wax precipitation are not able to represent the properties of the n-paraffins correctly when part of a petroleum fluid because the Van der Waals mixing rules cannot handle hydrocarbon mixtures containing more than one pseudo-component distribution. The proposed mixing rules for ideal or athermal solutions described above can therefore be applied to improve wax thermodynamic modelling.

Wax models depend on knowing the concentration of n-paraffins in the fluids to quite high carbon numbers, typically C80. This means that the aromatic-paraffinic blending ratio cannot be used in this case as an adjustable parameter. Furthermore, the n-paraffins are well-defined chemical compounds, the properties of which are known and fixed. However, the present invention can be applied if three generic component distributions are used.

The n-paraffin concentrations are either directly measured or estimated for wax studies, and so cannot be treated as model variables. The remainder of the fluid may then be represented as a blend between generic aromatic components and generic aliphatic components. The aliphatic components represent the non-aromatic part of the oil without the n-paraffins. The aromatic-aliphatic blending ratio then becomes the adjustable parameter that has the same role as the aromatic-paraffinic ratio in the version of the method with two generic component distributions.

In calculating the properties of the fluids, the contribution from the n-paraffins has to be included but that is always possible because the concentration and properties of the n-paraffins for wax studies are fixed.

A further application of the present invention relates to the problem of allowing for contamination by drilling mud.

When bottom-hole samples of reservoir fluid are taken from test wells, the sample is often contaminated with the organic constituents of drilling mud. The present invention can provide a faster and more reliable method of predicting the properties of the uncontaminated reservoir fluid from the contaminated sample.

The procedure is to characterise the contaminated sample according to the present invention. For the purposes of this exercise there is no need to group the SCN cuts into pseudo-components as using the full SCN distribution is more accurate and there is no necessity to reduce the number of components to save computing time.

It is also necessary to have an analysis of the mud fluid that is causing the contamination. The mud fluid is also characterised using the generic components method, again without grouping components into pseudo-components.

As with current methods, the amount of mud fluid that has contaminated the sample is estimated either by comparing the shapes of the SCN distributions that have been measured, or else by reference to markers in the mud fluid.

Using the present invention, it is then a simple step to estimate the composition and properties of the uncontaminated fluid. The amounts (either by mole or by mass) of the generic pseudo-components in the mud fluid are subtracted from the amounts of the corresponding generic components in the contaminated fluid in the right proportion to reflect the amount of mud fluid that has entered the sample. The result gives the composition of the uncontaminated fluid; the changes in the aromatic-paraffinic component ratios in the uncontaminated fluid is the mechanism whereby its properties are predicted.

In comparison, the conventional method suffers from the problem that the properties of the components used to model the uncontaminated oil and the mud fluid may not be the same, and may also be different from the properties of the pseudo-components of the uncontaminated fluid. The need to predict the properties of the pseudo-components of the uncontaminated fluid is an additional and potentially difficult aspect of the conventional characterisation approach that is absent from the generic component approach.

The present invention may also be used to assist with allocation agreements.

Since many oil-industry development involve shared facilities with commingled production, allocation agreements become an important contractual issue as large financial assignments are influenced by the methodology adopted. The present invention offers the basis for rationalising allocation agreements. If the sources of production, typically well-head fluids, are all characterised using the generic component method, the measured flow-rate can be converted to a flow-rate of generic components using suitable measurements of phase flow-rates, phase densities etc. If a relative monetary value is then assigned to each generic component, the value of each source of production can be calculated on a common basis. A more sophisticated version might also involve characterising the fluid arriving at the receiving point using the same method. Naturally there will not be perfect agreement between the measurements owing to measurement and model errors. However, a reconciliation procedure could then be introduced, making due allowance for time elapsed between production at the well-head and arrival at the receiving point.

The modelling of polymers is a further example where the present invention may be applied.

Polymers have many properties that parallel those of crude oils. They consist of distributions of similar molecules but of differing length and hence molecular weight. Co-polymers are formed of molecules made up of varying ratios of two or more constituent monomers, but again of varying molecular weight. Such polymer systems can readily be modelled with a small number of pseudo-components that represent groups of large numbers of polymer molecules of broadly similar compositions and molecular weights. A cubic equation of state could be used to model the polymer mixture provided the mixing rules are adequate to describe the polymer solution. Experimentally, it is found that many polymers show mixture properties that can be described by the Flory-Huggins theory. A Flory-Huggins mixture is athermal in nature (see P. J. Flory: Principles of Polymer Chemistry, Cornell University Press, 1953), so the above mixing rules for an athermal solution (or more approximately for an ideal solution) would enable cubic equations of state and similar equations to be applied to processes involving polymers, facilitating design studies for polymer production and process control.

Further applications of the present invention are in the modelling of solubility, or of asphaltenes and naphthenic acids, for example.

Many substances can dissolve in petroleum fluids to different extents depending on the fluid. In many cases the degree of aromaticity is a major controlling factor. For example, it is known that the mutual solubilities of glycol (ethane-1,2-diol) and aromatic hydrocarbons can be an order of magnitude higher than for paraffinic hydrocarbons (see M. Riaz, et al.: J. Chem. Eng. Data, 56, 4342, 2011). The present invention can be used to model such cases because it allows the petroleum fluid to be modelled as a mixture of aromatic and paraffinic components owing to its mixing rules which predict physically realistic mixing behaviour. The solubility between hydrocarbons and some other substance can therefore be reproduced by adjusting the BIPs between the substance in question and the petroleum pseudo-components. The key advantage is that the BIP values for the aromatic pseudo-components may be different from those for the paraffinic pseudo-components. Not only does this technique allow one to account for the influence of the fluid's aromaticity on solubility but also it enables the model to simulate the effect that the dissolved hydrocarbon fluid can be enriched in aromatic fractions.

Asphaltenes are a particular case where the aromaticity of an oil affects solution behaviour. Asphaltenes are the most polar and heavy constituent of crude oils; they are of engineering importance because they can deposit from oil due to changes in pressure, temperature or composition causing major disruption to production (see J. X. Wang, J. L. Creek, J. S. Buckley: Screening for Asphaltene Problems, SPE 103137, 2006). Asphaltenes are solubilised in oils by the aromatic components and another group of constituents called resins. The present invention allows an asphaltenic oil to be represented by a mixture of pseudo-components representing the paraffinic, naphthenic, aromatic, resinous and asphaltenic fractions of the oil. Each category of component can be assigned different pure-component properties while the mixing rules ensure correct mixing behaviour is predicted. If necessary BIPs can be introduced between the asphaltenes and the other hydrocarbon components, the justification being that the high polarity of asphaltene molecules suggests that they will not form an ideal or athermal solution with other hydrocarbon molecules.

Another category of component that occurs in petroleum fluids is naphthenic acids. These are naturally occurring constituents of oils that have one or more carboxyl groups. They are surfactants that can also partition into a saline aqueous phase where they can form natural soaps and scums, often with serious operational consequences. When dissolved in oil, naphthenic acids can behave in a highly non-ideal way as the carboxyl groups are extremely polar and form double hydrogen bonds between one acid molecule and another. The aromaticity of the oil can be expected to influence the solubilities of naphthenic acids in that oil, and hence their tendency to partition into the aqueous phase. Again, the present invention allows this effect to be modelled because the oil can be explicitly represented as a mixture of paraffinic and aromatic pseudo-components. BIPs can be introduced, if necessary, between the hydrocarbon pseudo-components and the naphthenic acids, and again the aromatic and paraffinic pseudo-components can be differentiated.

1. A method of characterising a mixture comprising a plurality of hydrocarbons, said method comprising:
obtaining a set of predetermined pseudo-components, each pseudo-component representing a respective group of hydrocarbons;
obtaining physical data about the hydrocarbon mixture; and
determining from the data the amount of each pseudo-component considered to be present in the mixture.

2. A method as claimed in claim 1, wherein the set of predetermined pseudo-components comprises a distribution of pseudo-components according to the hydrocarbon carbon number.

3. A method as claimed in claim 2, wherein the set of predetermined pseudo-components comprises a distribution of single carbon number cuts.

4. A method as claimed in claim 2, wherein the set of predetermined pseudo-components comprises a distribution of single carbon number cuts grouped together into a number of pseudo-components, preferably 4-7 pseudo-components, with each pseudo-component comprising more than one single carbon number cut.

5. A method as claimed in claim 1, wherein two or more distributions of predetermined pseudo-components are used.

6. A method as claimed in claim 5, wherein two distributions of predetermined pseudo-components are used and the two distributions of pseudo-components correspond to an aromatic distribution and a paraffinic distribution, respectively.

7. A method as claimed in claim 1, wherein the physical data is obtained by at least one of gas chromatography, TBP distillation, and ASTM D86 distillation.

8. A method as claimed in claim 1, wherein the amount of each pseudo-component present in the mixture is determined by adjusting blend ratios of the pseudo-components until they reproduce physical properties of the mixture determined from the physical data.

9. A method as claimed in claim 8, comprising averaging the properties of the constituent pseudo-components in order to predict the physical properties of the mixture.

10. A method as claimed in claim 1, further comprising modelling the properties of the mixture using an equation of state.

11. A method as claimed in claim 10, wherein the modelling comprises using a mixing rule for the equation of state adapted for an athermal or an ideal solution, preferably where the mixing rule is adapted for an athermal and/or an ideal solution.

12. A method as claimed in claim 10, wherein the modelling comprises using a mixing rule for the equation of state adapted for polar components with binary interaction parameters.

13. A method as claimed in claim 1, further comprising simulating blending a plurality of mixtures together, each mixture having been characterised using the method of claim 1, wherein the mixtures are all characterised using the same pseudo-components.

14. A method as claimed in claim 1, wherein the mixture is a petroleum fluid.

15. A method of modelling: wax precipitation in a fluid, contamination from drilling mud in a petroleum sample, polymers, solubility, asphaltenes or naphthenic acids, wherein the method comprises using a method as claimed in claim 1.

16. A method of determining allocation agreements for a group of oil wells comprising using a method as claimed in claim 1.

17. A computer program configured to perform the method of claim 1 when executed on a computer.

18-20. (canceled)

21. A method of characterising a mixture comprising a plurality of hydrocarbons, said method comprising:
obtaining physical data about the mixture;
defining two or more distributions of pseudo-components, each distribution representing a respective group of hydrocarbons, and each pseudo-component of each distribution representing a respective sub-group of that distribution;
determining from the data the amount of each pseudo-component of each distribution considered to be present in the mixture; and
applying a mixing rule to the pseudo-components, wherein the mixing rule is adapted for an athermal and/or an ideal solution.

22-33. (canceled)

34. A computer program for characterising a mixture comprising a plurality of hydrocarbons, said computer program being configured to perform the following steps when executed on a computer:
obtain physical data about the mixture;
define two or more distributions of pseudo-components, each distribution representing a respective group of hydrocarbons, and each pseudo-component of each distribution representing a respective sub-group of that distribution;
determine from the data the amount of each pseudo-component of each distribution considered to be present in the mixture; and
apply a mixing rule to the pseudo-components, wherein the mixing rule is adapted for an athermal and/or an ideal solution.

35-36. (canceled)
